(19) United States
(12) Patent Application Publication
(10) Pub. No.: US 2009/0209025 A1
(43) Pub. Date: Aug. 20, 2009

(54) HIGH SOLID THERMOPHILIC ANAEROBIC DIGESTER

(75) Inventors: Reinhard Goschl, Pitten (AT); Teck Fook Edwin Khew, Singapore (SG)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(73) Assignee: IUT GLOBAL PTE LTD., VALLLEY POINT (SG)

(21) Appl. No.: 12/294,114

(22) PCT Filed: Mar. 30, 2006
(86) PCT No.: PCT/SG06/00077

§ 371 Date: Sep. 23, 2008

(51) Int. Cl. A62D 3/02; C12M 1/00

(52) U.S. Cl. 435/262.5; 435/303.2

(57) ABSTRACT

A device for digesting sludge anaerobically, comprising a digesting tank (100) having an upper region (104) and a lower region (105), and a reaction chamber (107) for converting raw sludge into matured sludge, an inlet (112) for introducing sludge (109) into the digesting tank, at least one transfer pipe (120) for channelling sludge from the lowe region of the digesting tank to the upper region of the digesting tank, said at least one transfer pipe being arranged within the digesting tank and having at least a part of its length thereof arranged within the reaction chamber so that least one transfer pipe is in contact with sludge moving through the reaction chamber, thereby resulting in heat transfer from sludge moving in at least on transfer pipe to sludge in the reaction chamber, and an outlet (114) arrange at the lower region of the digesting tank for discharging matured sludge (116) from the digesting tank.

The present invention relates generally to the field of waste treatment, and more particularly to a device, a process and a system for the anaerobic digestion of organic sludge.

### BACKGROUND OF THE INVENTION

Anaerobic digestion is a commonly used process for the treatment of organic waste material. Many types of organic wastes can be treated by anaerobic digestion, including agricultural, domestic and industrial wastes. One of the chief objectives in carrying out digestion of organic waste is to convert sludge solids into a clean effluent suitable for discharge into the environment. The production of methane, a combustible fuel, as a by-product of the anaerobic digestion process is also an important aspect of the operation of an anaerobic digestion plant which helps to lower the running costs of the plant. As organic waste is produced in large quantities from both industrial, commercial, and agricultural cities, the treatment of organic wastes via anaerobic digestion represents an economically attractive method of waste disposal/treatment and recycling.

As compared to aerobic digestion process, anaerobic digestion is generally more efficient at removing sludge solids and therefore produces less sludge than aerobic digestion (see U.S. Pat. No. 4,885,094). However, anaerobic digestion typically requires long residence times to allow the anaerobic bacteria time to breakdown the organic material in the sludge (see U.S. Pat. No. 5,637,219). Based on considerations of efficiency, batch anaerobic digesters usually operate viably on a large scale requiring large foot print, whereas digesters which operate continuously are preferred as they produce a steady supply of methane gas and bio-compost and operate in smaller compact sites.

Continuous anaerobic digesters are classically modelled either after the one-stage continuously-stirred tank reactor (“CSTR”) or the plug-flow tank reactor (“PFTR”). The former is usually used to treat sludge containing low levels of sludge solids (typically less than 10% dry matter) while the latter is commonly used to treat sludge with high solid content (see U.S. Pat. No. 6,673,243). In a plug flow reactor, sludge is directed through the digester from inlet to outlet in a sequential manner, without any intermittent mixing with fresh undigested sludge. By providing a sufficiently long residence time in the reactor, sludge is ideally completely digested upon reaching the outlet.

The type of anaerobic bacteria used for digesting sludge in the digester determines the optimal temperature range for the digester to operate efficiently. Mesophiles prefer operating temperatures of about 20° C. to about 45° C., whereas thermophiles prefer operating temperatures of about 50° C. to 65° C. The yield of methane drops if the operating temperature falls outside the optimal range. Digesters operating at thermophilic temperature range has the advantage of shorter residence times, but requires expensive energy input to maintain the temperature elevation of about 30° C. to 40° C. above ambient or room temperature.

For this reason, thermophilic digestion is often considered to be economically unattractive for the treatment of sludge because the heat source required to operate the digester is seldom justified by the economic benefit derived from the production of raw methane gas (which contains corrosive components) and compost. Various attempts have been made to address problems in implementing the anaerobic digestion of waste in the past.

U.S. Pat. No. 6,673,243 discloses a plug flow anaerobic digester comprising a sequentially arranged series of three chambers each providing an environment suitable for anaerobic microorganisms to efficiently digest sludge. The volume of each chamber is designed to control the relative residence time of the sludge at different stages of digestion. As the initial stages of fermentative and hydrolytic digestion are carried out faster than the later stages of acetogenesis and methanogenesis, the first chamber is designed to provide shorter residence time than the second and the third chambers. No external heating is provided, meaning that influent sludge is treated at ambient temperature dependent upon the climate.

U.S. Pat. No. 6,929,744 describes a pilot-scale digester comprising an inner cylindrical tower arranged within an outer cylindrical tower, thereby defining a central cylindrical chamber and an outer annular chamber. Raw sludge is incubated in a closed vessel for 3 days at 35° C. introduced into the annular chamber at the bottom of the digester, and then pumped upwards until it overflows into the central chamber with the aid of a flow distributor.

US Patent Application No. 2005/0077238 describes an egg-shaped anaerobic digester having draft tubes arranged within the digester to enable sludge to be transported from the top section and the bottom section of the digester to the middle section. The draft tubes provide control of the digestion process to accommodate the formation of scum and foam, which may be detrimental to mixing within the digester if not managed properly.

U.S. Pat. No. 6,632,362 describes a multi-stage anaerobic digester having cross-sectional grids to separate floating media at different phases of digestion along the length of the digester. Raw sludge is fed to the top of the digester, which gradually descends down the digester to be digested. Concentrated digested sludge sinks to the bottom of the digester to be discharged. Methane produced is scrubbed in a methane separator, and the pure methane obtained is used to power a boiler which in turn is used to heat the raw sludge. However, the use of scrubbed methane for heating the raw sludge is not economical, since operation of the scrubber is costly and the scrubbed methane can be sold.

It is an objective of the present invention to provide an alternative anaerobic sludge digester which addresses at least some of the drawbacks of all the above-mentioned prior art.

### SUMMARY OF THE INVENTION

According to a first aspect of the present invention, a device for digesting sludge anaerobically is provided. The device comprises a digesting tank having an upper region and a lower region, and a reaction chamber for converting raw sludge into matured sludge. The digesting tank has an inlet for introducing the raw sludge into the digesting tank, and an outlet arranged at the lower region of the digesting tank for discharging matured sludge from the digesting tank. At least one transfer pipe is present for channelling sludge from the lower region of the digesting tank to the upper region of the digesting tank. The transfer pipe(s) is arranged within the digesting tank and has at least a part of its length thereof arranged within the reaction chamber so that it is contacted with sludge moving through the reaction chamber, thereby resulting in heat transfer between sludge moving through the reaction chamber and sludge within at least one transfer pipe.

The second aspect of the invention is directed to a process for treating sludge anaerobically comprising introducing raw sludge into a device according to the invention. The sludge is passed through the reaction chamber for a period of time sufficient for the slurry to be anaerobically digested. A portion of the sludge is channelled from the lower region of the digesting tank to the upper region of the digesting tank via at least one transfer pipe present in the device. Matured sludge is discharged from the digesting tank via the outlet.

A third aspect of the present invention is directed to a system for digesting sludge anaerobically. This system comprises screening means for removing inorganic material from raw sludge, shredding means for reducing the size of the raw sludge, and a device for digesting the raw sludge anaerobically in accordance with the present invention.

The device of the present invention presents several advantages over prior art digesters. Firstly, the transfer pipes arranged within the digesting tank helps to lower net energy requirements of the digester and to keep temperature nearly uniform throughout the whole digester by facilitating the transfer of heat from raw sludge to the matured sludge within the digester. In this manner, the preheated raw sludge through heat exchange provides heat to the mature sludge as it flows up the digester and exits at the top of the digester at a temperature suitable for commencing anaerobic digestion in the digesting tank. Additionally, a wide range of organic waste, including discarded food material, animal manure, abattoir waste, vegetable waste, horticultural crop residues, industrial organic wastes, sewage sludge and source-separated household organic waste, etc. can be completely processed into compost which can be used as fertilizers, thereby facilitating the reutilization and recycling of carbon back to earth. No waste water is being discharged from the system as all waste water generated is completely recovered and recycled and reused. Structural material, which is thoroughly mixed with the digested sludge to assist in the aeration and maturation of the digested sludge during the composting process is also recovered and reused. These advantages help to lower net material requirements and thereby lower operating costs. Biogas produced by the anaerobic digestion of waste can be recycled or utilised for heat generation (such as municipal district heating) or for driving generators in a power grid. Additionally, no internal stirring mechanism is required for the digestion to occur. This ensures a low maintenance, highly efficient, continuously operating digester which requires minimal maintenance down time. Accordingly, the invention not only facilitates an environmentally friendly treatment of organic waste material, it also attempts to make the process economically self-sustainable, providing renewable energy and inhibiting the formation of green house gases like methane and carbon dioxide.

In the context of the specification, the term “raw sludge” or “raw” slurry” refers to untreated or undigested sludge that is being introduced into the digesting tank. The term “raw” does not exclude the possibility that the sludge is pre-treated, such as shredding to reduce the average size of the sludge, or heat treated to reduce pathogens in the sludge. The term “mature sludge” or “matured sludge” is used interchangeably with the term “treated sludge” or “digested sludge” which refers to sludge that has gone through at least one pass through the reaction chamber of the digesting tank, and is thus at least partially anaerobically digested. The term is therefore not restricted to sludge which has been completely digested.

The device of the present invention comprises a digesting tank having a reaction chamber within which anaerobic digestion of the sludge occurs. The digesting tank comprises any vessel having appropriate dimensions for housing a reaction chamber that can continuously process the sludge in a single stage. A continuous process is usually favoured, since the sludge is processed continuously to produce a steady supply of methane and compost. For example, the digesting tank may take the form of a substantially vertically oriented reactor column.

The digesting tank comprises an upper region and a lower region. The upper region refers to any part of the digesting tank located above its middle, and correspondingly, the lower region refers to any part of the digesting tank located below the middle. The digesting tank has an inlet through which raw sludge is introduced into the digesting tank, and an outlet arranged at the lower region of the digesting tank for discharging matured sludge from the digesting tank and which is preferably insulated to reduce heat loss to a minimum. The inlet may be arranged at either the lower region or the upper region, or both, depending on the required design.

The reaction chamber arranged within the digesting tank serves to provide an environment that is suitable for the anaerobic bacteria to digest the sludge. Depending on the amount of sludge that is to be treated, the dimensions of the digesting tank may be selected to accommodate the amount of sludge to be digested. The volume of the reaction chamber is typically selected to control the relative residence time required to digest the sludge. These dimensions are selected such that the sludge is anaerobically digested either with a single-pass or with several passes through the digesting tank. As the reaction chamber is accommodated within the digesting tank, the dimensions of the reaction chamber usually determines the dimensions of the digesting tank. The reaction chamber may comprise a segment of the digesting tank, or it may comprise a separately defined compartment within the digesting tank.

In the present invention, at least one transfer pipe, or more preferably, a plurality of transfer pipes are present for channelling sludge from the lower region of the digesting tank to the upper region of the digesting tank. Each transfer pipe is arranged within the digesting tank, either mounted to the internal walls of the digesting tank or otherwise, and has at least a part of its length thereof arranged within the reaction chamber. The purpose of so doing is to enable down-moving sludge that is moving through the reaction chamber (hereinafter used interchangeably with the term ‘maturing sludge’ or ‘digesting sludge’) to come into contact with the transfer pipe. As the sludge that is moving down through the reaction chamber will usually lose some heat as it moves down the reaction chamber, in order to maintain an optimum anaerobic digestion temperature, the mixed sludge can be pre-heated before it is introduced into the reaction chamber transfer pipe. By heating the mixed sludge to a higher temperature than that of the sludge in the reaction chamber before, sludge in the reaction chamber is at a lower temperature than the mixed sludge. A temperature gradient thus exists between the cooler down-moving sludge and the warmer raw/mixed sludge moving up through the transfer pipe. This temperature gradient results in heat transfer from the warm mixed sludge moving up the transfer pipe(s) to the down-moving maturing sludge within the reaction chamber. In this manner, maturing sludge in the reaction chamber is maintained at a consistent temperature by the heated mixed sludge. On the other hand, as the heated mixed sludge loses heat to the maturing sludge, its temperature falls throughout its transit in the transfer pipe. When the raw sludge is discharged from the transfer pipe at the upper region, it's temperature would have fallen to a predetermined temperature suitable for thermophilic digestion to occur. Source of heat for raising the temperature of the mixed sludge prior to transmitting it into the transfer pipe may come from a heat exchanger where heat is provided by gas engines driven by produced methane, for example.

In one embodiment, at least one transfer pipe is adapted to facilitate the transfer of heat from the sludge moving up at least one transfer pipe and sludge moving down the reaction chamber, thereby improving the efficiency of heat transfer to the sludge in the reaction chamber. For example, the transfer pipe may include fins on its external surface to increase the available surface area for contact with the down-moving sludge or at least one transfer pipe may assume any suitable configuration to maximise contact with the sludge in the reaction chamber, including a straight pipe or a coiled pipe configuration.

An actuating means may be provided for pumping the sludge through at least one transfer pipe. An example of the actuating means includes a screw pump, piston pump, diaphragm pump etc. The transfer pipe discharges the mixed sludge at one or several points in the upper region of the digesting tank, optionally with the aid of a distributor means for achieving even distribution of the mixed sludge in the reaction chamber. Discharged mixed sludge then enters the reaction chamber and commences its journey down the digester, typically taking between 15 to 21 days or more.

Upon reaching the bottom of the reaction chamber, raw organic material in the mixed sludge would have become digested, i.e. complex organic molecules in the raw sludge is broken down from a complex form to a simpler form, thereby converting the mixed sludge into mature sludge. Mature sludge leaves the digesting tank from the outlet located at the lower region of the digesting tank. A major portion of the mature sludge is recycled into the digesting tank while a small portion is extracted for composting and maturation to produce high grade pathogen free bio-compost.

Any anaerobic microorganism may be used for facilitating anaerobic digestion. Common types of bacteria used for anaerobic digestion includes hydrolytic bacteria, fermentative bacteria, methanogenic bacterium, and acetogenic bacterium. Specific examples of bacteria include methanobacter formicicum, methanobacter soehngenii, methanobacter ruminatium, methanococcus mazei, vanielli, methanosarcina methanica, and methanosarcina thermophilia. Common mold, and fungi may also be used for facilitating the digestion.

Under most conditions, no deliberate addition of bacteria is necessary. By mixing part of the mature sludge with raw sludge, native bacteria present in the mature sludge is introduced into the raw sludge and is able to work the raw sludge under conditions to which the bacteria is already adapted. In order to establish an initial bacterial population in the reaction chamber of the digester, organic waste is fed to the digester at a flow rate necessary to achieve an initial residence time of about 21 days.

In order for anaerobic digestion to occur, oxygen concentration in the reactor is kept at a minimum preferable at zero. This is done for example by ensuring that the digesting tank is hermetically sealed, and preferably kept under a slight vacuum. This is achieved by extracting gases from the top of the digesting tank (where produced gases accumulate). In addition, it may be possible, in one embodiment, to have a digesting tank in which the reaction chamber is adapted to maintain a slight negative pressure. This may be achieved by hermetically sealing the digesting tank with the aid of a flat or dome-shaped lid having incorporated therein a gas outlet from which gases produced from the digestion may be continuously removed. Alternatively, it is also possible to continuously introduce an inert gas, such as nitrogen, into the digesting tank in order to reduce the amount of oxygen.

Prior to adding raw sludge into the digesting tank, the raw sludge is preferably mixed with mature sludge in order to introduce anaerobic bacteria into the raw sludge. The mixing can be carried out in any suitable way, such as stirring the mixture in a mixing tank or channelling the mixed sludge through a sludge mixer. The mixing can be carried out either within the digesting tank or outside of the digesting tank. To reduce the loss of heat, mixing may be carried out within the digesting tank, for example.

In one embodiment, the mixing means comprises a mixing region arranged in the lower region of the digesting tank, the mixing region being adapted to receive matured sludge from the reaction chamber and raw sludge from the inlet. The introduction of raw sludge into the lower region of the digester enables the raw sludge to be ‘seeded’ and mixed with thermophiles and matured sludge, thereby forming a mixed sludge. After mixing, the mixed sludge maybe transmitted through one or more transfer pipes to the upper region of the digesting tank. Alternatively, instead of being directly transmitted up the transfer pipe, this ‘seeded’ raw sludge may be withdrawn from this mixing region via one or more screw pumps (through which thorough mixing occurs) and then heated in a heat exchanger before feeding into the transfer pipes to the upper region of the digester. This feature not only helps to avoid the process of separate pre-mixing before feeding into the digester, but by spiking the temperature of the mixed sludge above the temperature of the digesting sludge in the reaction chamber, heat is transferred from the mixed sludge to the digesting sludge, thereby helping to maintain digesting temperature in the reaction chamber.

Alternatively, the mixing means may comprise at least one screw pump, or more preferably, a plurality of screw pumps, taking suctions from the mixing region in the digesting tank, wherein both matured sludge and raw sludge are extracted from the digesting tank. The outlet of the screw pump is connected to the transfer pipe to transmit the mixed sludge to the top of the digesting tank where digestion commences.

The digestion of sludge produces biogas, a large percentage of which comprises methane gas. Methane gas is vented from the digesting tank via an outlet arranged at the upper region. In this context, the term biogas refers to the mixture of gases extracted from the outlet of the digesting tank, and is not limited to gases produced from anaerobic digestion alone. These gases are derived from a myriad of processes occurring within the reaction chamber, including, respiration, anaerobic fermentation and the production of alcohols and hydrogen by various types of bacteria acting on the sludge.

The other aspects of the invention are directed to a process and a system for treating sludge anaerobically. The process comprises introducing raw sludge into a device according to the first aspect of the invention, passing the raw sludge through the reaction chamber (lower portion) for a period of time sufficient for the raw sludge to be exposed to mature sludge and therefore seeded with thermophiles. Prior to being introduced into the upper region of the digesting tank where digestion commences, the raw sludge is mixed with digested mature sludge in a screw pump to form a mixed sludge. The purpose of this mixing is to thoroughly mix the native anaerobic bacteria (thermophiles) into the raw sludge, thereby rendering it suitable for anaerobic digestion in the digesting tank when introduced to the upper portion of the digester.

Anaerobic digestion typically involves three basic steps. The first step involves preparation of the organic fraction of the solid waste for anaerobic digestion and usually involves receiving sorting separation and size reduction. The second step involves the addition of moisture and nutrients, blending, pH adjustment to about 6.7, heating the slurry and anaerobic digestion in a reactor with continuous flow in which the contents are well mixed for a period of time varying from 15 to 21 days. The 3^{rd} step involves capture, storage and if necessary, separation of the gas components evolved during the digestion process. The fourth step is the composting and maturation of the digested sludge.

Design considerations in the process of the invention includes the size of the shredded raw sludge, extent of mixing, percentage of solid organic matter in the raw sludge. Other important factors to be considered include hydraulic residence time and raw sludge loading rate.

One feature in the process of the invention is the channelling of mixed sludge into at least one transfer pipe to be transferred from the lower region of the digesting tank to the upper region of the digesting tank. At least one transfer pipe has a section of its length thereof arranged in the reaction chamber of the digesting tank. As the sludge moving through the reaction chamber comes into contact with the transfer pipe containing heated mixed sludge, the temperature gradient results in heat transfer thereby ensuring a uniform optimum operating temperature within the digester which can be monitored and controlled thereby minimising the energy used in the whole digesting process.

Depending on the heat exchange desired as well as the size of the digesting tank, a plurality of transfer pipes may be installed in the digesting tank. For example, any number of transfer pipes ranging from 2, 3, 4, 5 or more transfer pipes may be installed in the digesting tank. In order to facilitate heat transfer, the pipes are preferably made of high conductivity material which is at the same time corrosion-resistant. Examples of such a material include stainless steel alloys and copper.

In most digesters, anaerobic bacteria that are used for digesting the sludge determine the optimum temperature for the digester to operate at peak efficiency. For thermophilic digestion to occur, the temperature range is typically between about 50° C. to about 65° C. Climate changes may lead to variations in the temperature at which the sludge is being treated. If such changes occur such that conditions within the reaction chamber falls outside this thermophilic temperature range, then methane yield may drop. For this reason, an important consideration in the design of the digester is the efficient control of temperature at which sludge is being processed in the reaction chamber. More preferably, operating temperatures within the reaction chamber are kept in the range of about 49° C. to 57° C. for thermophilic anaerobic digestion to occur. In cold climates, a portion of the biogas obtained from the digestion is used to run hot water boilers in order to maintain the control of this temperature range. In order to keep the anaerobic bacteria functioning effectively, pH of the sludge is preferably kept in the range of about 6 to 8.

One possible approach to obtain good reactor performance is to ensure that the reaction chamber space within the digesting tank is occupied by as much biodegradable material as possible. This means that non-biodegradable material which is not digestible, and thus does not produce any methane, should as far as possible be removed from the raw sludge prior to digestion. To optimise the processing capacity of the digesting tank, non-biodegradable material such as metals, plastics, stone, and wood may be mechanically separated out. Separation can be carried out based on differences in size, weight and density. A variety of mechanical separation methods may be used for this purpose, including screening, air separation and pneumatic separation or a combination of all three. Screening is a preferred method for removing the inorganic materials and may be carried out via mechanical, optical separation or flotation separation.

In one embodiment, screening comprises a rotary screen and a shredder. Preferably, the rotary screen has a diameter of between about 140 to about 160 mm, more preferably about 150 mm; and the shredder then reduces the waste to a diameter of between about 14 mm to 16 mm. For example, trommels and vibrating screens may be used to reduce and remove unwanted inorganic articles from the sludge. Ferrous materials may be separated with the aid of an electromagnet.

Prior to adding the sludge to the digesting tank, it may be advantageous to reduce the size of sludge being processed and thereafter form a slurry/sludge mixture therefrom. The objective of size reduction is to provide as large a surface area as possible for digestion and to obtain a final product compost that is reasonably uniform in size and texture and therefore ensuring its miscibility with soil and earth as a planting media. One way of achieving this is to shred the sludge into an average size of less than 50 mm, preferably less than 30 mm and most preferably less than 20 mm. Subsequently, water is added to the shredded sludge to form a slurry mixture. In one embodiment, the shredded sludge is mixed with water to form a raw slurry/sludge having a consistency of about 10% to about 20% of dry solid content. Shredding may be done any time before the sludge is introduced into the digesting tank, but preferably carried out after screening. Any conventional shredding equipment may be used for this purpose, such as two-stage coarse-fine low-speed shredders, as well as single-stage shredders with screen and recycle of oversized material.

Anaerobic bacteria may be introduced into the raw sludge by adding cultured bacteria to the raw sludge prior to introduction into the digester. Alternatively, the raw sludge is mixed with matured sludge leaving the outlet of the digesting tank to form a mixed sludge. The advantage of the latter over the former is that the matured sludge contains bacteria native to the digesting tank which are already adapted to the conditions within the digesting tank, and should therefore be efficient in digesting the raw sludge. The mixed sludge is transported to the upper region of the digesting tank via the transfer pipes, so that the mixed sludge is subjected to anaerobic digestion in the digesting tank. In one embodiment, the raw sludge is mixed with digested sludge in the ratio of about 1 part raw sludge to 9 parts digested sludge.

Matured sludge that has been discharged from the digesting tank is composted to further break it down into dry, manageable compost. The composting process may comprise laying the digested sludge in the open to be dried, or drying the digested sludge in aerating units. Preferably, composting comprises aerating and humidifying the digested sludge. To improve the composting process, the mature sludge may be mixed with wood chips prior to aeration in order to increase the porosity of the sludge.

Prior to composting, it is possible to extract water from the digested sludge in order to recover and recycle the bacteria-rich water. So doing also enables the digested sludge to dry faster. In one embodiment, dewatering is carried out until the dry solid content in the fermented sludge is about 25% to about 30%. Dewatering is typically done by mechanically squeezing the digested sludge, for example in a screw press or any other equivalent equipment.

These aspects of the present invention and the advantages will be more fully understood in view of the following description, drawings and non-limiting examples.

### BRIEF DESCRIPTION OF THE DRAWINGS

In order to understand the present invention and to demonstrate how it may be carried out in practice, preferred embodiments will now be described by way of non-limiting examples only, with reference to the accompanying drawings, in which:

FIG. 1 shows an embodiment of the device according to the invention in which the recycle stream containing mature sludge is mixed with a raw sludge stream outside of the digesting tank. One transfer pipe is present in the digesting tank for transmitting mixed sludge up the digesting tank.

FIG. 2 shows another embodiment in which two transfer pipes are present in the digesting tank.

FIG. 3 shows yet another embodiment of the device according to the invention in which raw sludge is introduced into the digesting tank to be mixed with matured sludge at a mixing region within the digesting tank.

FIGS. 4, 5 and 6 shows a various views of one embodiment of the device according to the invention in which four transfer pipes are present. In this embodiment, mixing of raw sludge with matured sludge occurs at a mixing region within the digesting tank, as well as in a mixing device outside the digesting tank.

FIGS. 7 and 8 depicts the side and perspective view of a screener.

FIG. 9 shows a simplified flow diagram of the process according to the invention.

FIG. 10 shows a simplified flow diagram illustrating the various units comprised in the system according to the invention as well as the processes carried out in such a system.

### DETAILED DESCRIPTION OF PREFERRED EMBODIMENTS

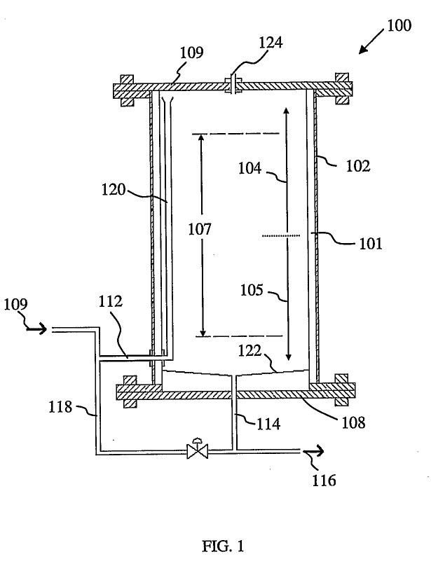

FIG. 1 shows a first embodiment of the device according to the invention. In this embodiment, the device 100 comprises a digesting tank 102 having an upper region as denoted by the arrow 104 and a lower region denoted by the arrow 105. Arranged within the digesting tank 102 is a reaction chamber 107 where anaerobic digestion of sludge takes place. A raw sludge stream 109 is introduced into the digester via inlet 112 located at the lower region 105 and leaves the digesting tank via outlet 114. A portion of the matured sludge is discharged via discharge stream 116 while the remaining portion of the matured sludge is recycled via recycle stream 118. Recycle stream 118 is combined with raw sludge stream 109 at inlet 112, thereby mixing mature sludge with raw sludge (hereinafter known as mixed sludge). This provides the raw sludge with the necessary anaerobic bacteria required for it to be digested. Mixed sludge enters the digesting tank 102 via the inlet 112. The inlet 112 is connected to a transfer pipe 120 that transports the mixed sludge to upper region 104. The transfer pipe 120 is arranged along the wall 101 of the digesting tank so that at least a portion of its length is located within the reaction chamber 107. The transfer pipe comes into contact with maturing sludge descending down the reaction chamber 107, thereby facilitating heat transfer between the maturing sludge in the reaction chamber 107 and the warm mixed sludge in the transfer pipe. This maintains the sludge in the digester at a uniform and constant temperature suitable for optimum thermophilic digestion to occur. Little temperature difference occurs between the upper and lower zones of the digester.

The mixed sludge is discharged from the transfer pipe and enters the reaction chamber 107, and begins its descent down the digesting tank 102. In the reaction chamber, bacteria break down complex biological molecules in the mixed sludge. In particular, carbon based substances are converted into methane. Methane and other gases released from the anaerobic digestion and other complex processes occurring in the reaction chamber rises to the upper region of the digesting tank and is evacuated via the gas outlet 124. Under ideal conditions, upon reaching the bottom of the digesting tank, the mixed sludge is completely digested/matured. The base 122 is sloped towards the centre so that the mature sludge is directed to the outlet 114, where it is once again partially discharged or recycled.

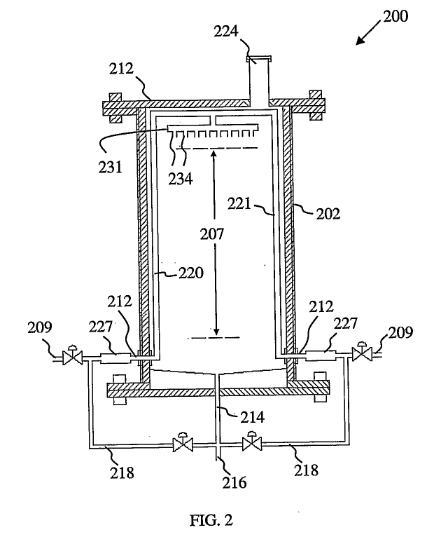

FIG. 2 shows a further embodiment of the invention in which device 200 comprises a first transfer pipe 220 and a second transfer pipe 221 arranged in the digesting tank. Each of the transfer pipes are connected to an inlet 212 located at the lower region of the device. Sludge enters the digester via inlet 212 and leaves the digesting tank via outlet 214. A portion of the matured sludge is discharged via discharge stream 216 while the remaining portion of the matured sludge is recycled via recycle streams 218. Recycle streams 218 are combined with raw sludge stream 209 and drawn up into sludge pumps 227. Apart from moving sludge up to the reaction chamber, the sludge pumps 227 also act as mixers where mature sludge is well mixed with raw sludge to form a mixed sludge.

Mixed sludge in each inlet 212 is transmitted via transfer pipe 220, 221 to a distributor 231 located at the upper region of the digesting tank. The distributor comprises a plurality of nozzles 234 which evenly distributes mixed sludge over the reaction chamber 207. In this embodiment, the gas outlet is arranged off the centre of the top 209 of the digesting tank 202.

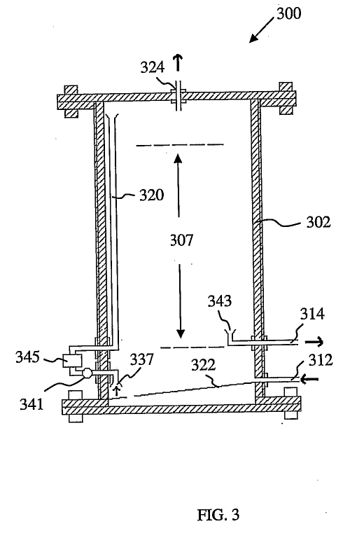

FIG. 3 shows another embodiment of the invention where the mixing between raw sludge and mature sludge occurs within the digesting tank. The device 300 comprises an inlet 312 and an outlet 314. Raw sludge entering the digesting tank 302 is mixed at mixing region 341 with oncoming mature sludge from the reaction chamber 307, thereby forming a mixed sludge. Mixed sludge is directed by the sloped base 322 to move towards suction inlet 337 of screw pump 341. The screw pump 341 provides additional mixing in the mixed sludge, and transmits the mixed sludge into a heat exchanger 345 where the mixed sludge is heated, and then transmitted back into the digesting tank, where the mixed sludge is delivered through transfer pipe 320 to the upper region of the digesting tank. Mixed sludge is gradually digested through the reaction chamber 307. A collection point 343 is provided above the inlet to channel some mature sludge into outlet 314 to be discharged.

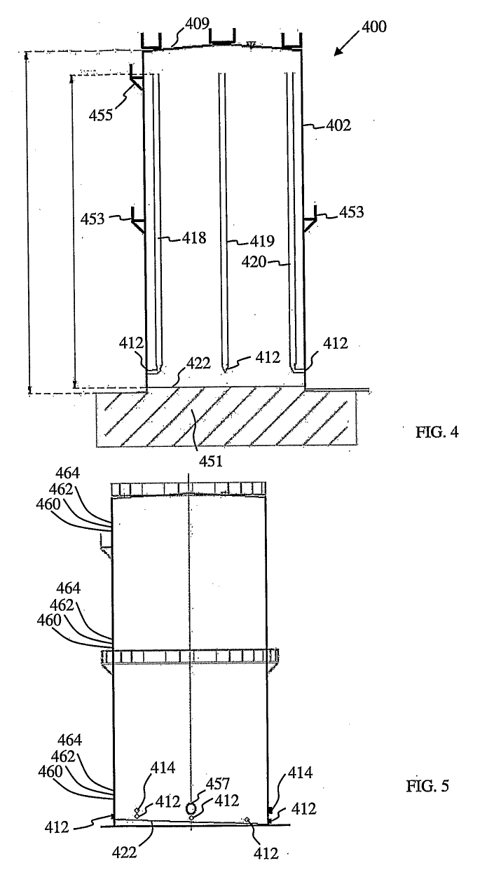

FIG. 4 shows a cross-sectional view of device 400 which is another embodiment of the invention in which digesting tank 402 comprises 4 transfer pipes 418, 419, 420, 421 (not shown in this diagram) each mounted internally within the digesting tank 402 and each connected to an inlet 412. The digesting tank 402 is seated on an enforced platform 451. The base 422 is sloped towards the centre, such that at the centre the base forms an angle of about 2° from the horizontal platform 451. Gangways 453, 455 arranged near the middle and near the top of the digesting tank, respectively, allows access to sampling points and to probes fitted into the digesting tank 402 to measure various operating parameters so that maintenance can be carried out.

FIG. 5 shows a side view of the device 400, illustrating the relative positions of the inlets 412, outlets 414 and manhole 457 allowing access into the digesting tank as seen on the exterior of the digesting tank 402. A plurality of test nozzles 460, temperature controls 462, and pressure controls 464 are arranged at the lower region, the middle, and the upper region of the digesting tank. The test nozzles 460 allows periodic extraction of sludge from the digesting tank for experimental test purposes.

FIG. 6 shows a top view of the digesting tank 402 as indicated in FIG. 4. Manholes 457 are provided in the top 409 of the digesting tank 402, and arranged near respective transfer pipes 420. A central gas outlet 424 is provided to extract gases produced in the course of digestion. Safety valves 466 are provided as a safety measure to prevent pressure build up within the digesting tank. In the event of pressure build-up, safety valves are triggered to release gases in the digesting tank. Subsequently a flaring system is triggered to flare off of the gases. Temperature controls 462 and pressure controls 464 are also provided at the top 409.

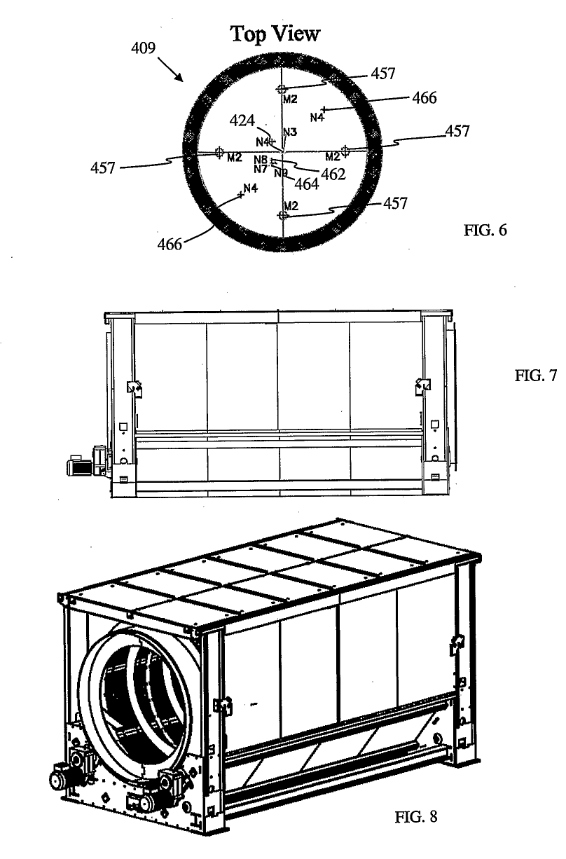

FIGS. 7 and 8 depict a screener that can be used in one embodiment of the invention. Any available generic types of screener that can provide a suitable screening size can be used.

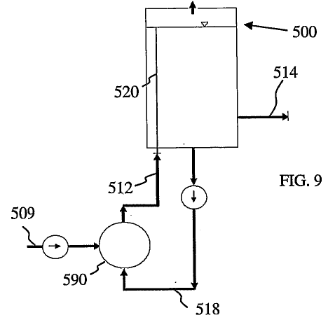

FIG. 9 shows a simplified process flow diagram according to the invention. A raw sludge stream 509 and a recycle stream 518 enters a digesting tank 500. The raw sludge stream 509 contains raw sludge that is to be anaerobically digested in the digesting tank, while the recycle stream 518 contains mature sludge containing live anaerobic bacteria. Upon mixing to form a mixed sludge, the live anaerobic bacteria in the mature sludge is introduced into the raw sludge. Mixed sludge is transferred via transfer pipe 520 to an upper region in the digesting tank where anaerobic digestion of the mixed sludge begins. The mixed sludge is allowed to reside within the reaction chamber of the digesting tank for a period of time sufficient for the any raw sludge present to be anaerobically digested, forming matured sludge. A portion of matured sludge is discharged for composting treatment via outlet 514, while the remaining portion of matured sludge is recycled into the digesting tank via recycle stream 518. In this embodiment, both recycle stream 518 and raw sludge stream 509 are heated via heat exchanger 590 near to or at thermophilic temperatures before entering the digesting tank.

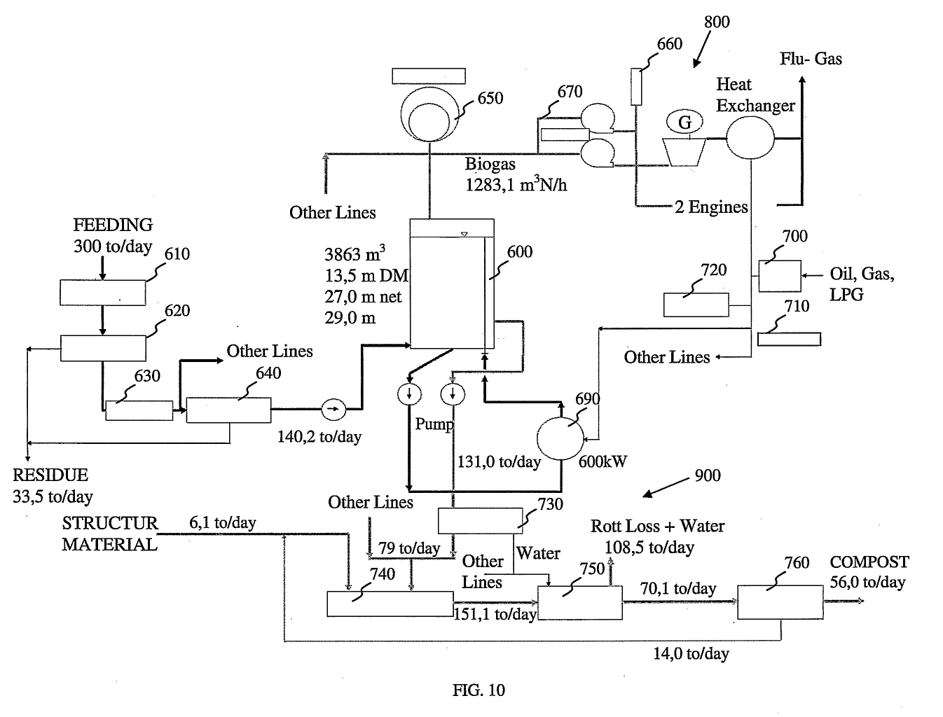

FIG. 10 shows a process diagram of another embodiment of the process according to the invention. The process is carried out on a system comprising the following: a device for carrying out anaerobic digestion of sludge according to the invention, screening means for removing inorganic material from a raw sludge, shredding means for reducing the size of the raw sludge, gas generator unit for combusting biogas produced from the digesting tank to produce electricity, a heat exchanger unit for transferring heat derived from the combustion to a portion of the raw sludge, a gas storage unit for storing the biogas, a composting unit for composting mature sludge discharged from the digesting tank, a dewatering means for removing water from the sludge, a mixing screw for mixing wood chips with the sludge that has been treated in the dewatering unit, and a composting device for converting the sludge that has been mixed with wood chips into compost.

Solid organic waste obtained from various collection points, such as farms, nurseries, food courts, factories, restaurants etc, are packaged into heavy-duty plastic waste collection bags. These waste collection bags typically carry up to 100 kg of solid waste and brought to the premises of the anaerobic digester. The bags are fed to a hopper that delivers the bags to an automated heavy-duty bag breaker unit 610 which breaks open the bags to expose the organic waste therein. The broken bag and its contents are conveyed via a series of conveyors to a screener 620. The screener 620 separates out the opened plastic bag and the inorganic material from the solid organic waste in order to maximise its organic content. The screened organic waste is then conveyed to an organic storage silo 630 to await further processing. The separated inorganic material, which may include metals, plastics, rubber sand and paper material, are conveyed to an inorganic storage hopper where it awaits discharge into bulk containers and then taken by trucks for recycling or disposal at landfills or incineration plants.

The organic waste is transferred via conveyors from the organic storage silo 630 to an organic shredder 640 which then shreds the organic sludge to a smaller size, preferably less than 20 mm. Water is added to the shredded sludge in order to provide a uniform slurry/sludge having between about 10% to 20% of dry solid content. The slurry is introduced through 1-6 inlets into the lower region of a digesting tank 600 where raw slurry is ‘seeded’ and mixed with thermophiles and matured sludge. In order for the raw slurry/sludge to be heated to a temperature suitable for thermophilic anaerobic digestion to take place (typically in the range of about 52° C. to 55° C.), the raw slurry/sludge is withdrawn from the bottom portion thru 1-6 outlets connected to screw pumps (in which thorough mixing occurs) and then heated in a heat exchanger to about 55° C. before feeding into the transfer pipes for delivery to the upper region of the digester. The heat exchangers can be supplied by hot water heated from the combustion of methane produced from the digester. As the heated mixed sludge moves up the transfer pipes, the heated raw slurry will transfer heat to the sludge in the digester therefore keeping the sludge at it optimum operating temperature as this sludge will loose heat as it moves from the upper portion to the lower portion. So the heated raw slurry will heat the mature sludge as it goes up the transfer pipe and keep it at about 52° C. or at any other temperature, preferably between about 52 to about 55° C. Anaerobic digestion commences when the mixed sludge is discharged from the transfer pipes and enters the reaction chamber. Conditions within the reaction chamber is adapted for anaerobic digestion to occur, e.g. temperature is suitably high and a slight vacuum is maintained to keep the concentration of gaseous oxygen low.

As digestion progresses, methane gas of approximately up to about 65% purity is produced. The digesting tank has a gas outlet arranged at the upper region through which the methane gas is collected and processed by a gas collection unit 800. The methane gas is extracted under vacuum and stored in gas storage units such as gas storage tanks 650. All the methane generated is used by gas generators to generate electricity and the heat from these generators are used to heat water for heating up raw sludge prior to digestion. A gas flaring safety system 660 is incorporated into the gas collection unit to consume the methane gas in the event that the gas engines are not operational.

Gas blowers 670 draw gas from the gas storage 650 and feed the gas into the injectors of gas generators 680. Gas generators 680 combust the methane in order to generate heat and electrical power. Electrical power is fed to a substation that is connected to a power grid, while heat is used for various applications, including preheating the raw sludge prior to feeding into the digesting tank via a heat exchangers 690, maintaining the digesting tank at temperatures required for thermophilic digestion, district heating, district cooling whereby the heat is used for the regeneration of liquid desiccants, or any other application requiring low temperature heat in the range of about 85° C. to 95° C. In some embodiments, heat is provided by gas fired or oil fired boiler 700 and a hot water system 710 in the event the gas generators are not operational. A cooling system 720 is included to prevent overheating of the gas generators.

The mixed sludge is fed continuously into the digesting tank 600. The residence time required for the raw sludge to be digested is approximately between 16 to 21 days. Digested sludge, also termed herein as digestate (matured sludge), is discharged continuously. In order to achieve a processing rate of 300 tons of food waste/restaurant waste per day, a digesting tank having an internal diameter of about 12 m and internal height of about 28 m may be used, for example. In this example, the digester may be operated at 52° C. and at a pressure of 0.05 bar.

A portion of the matured sludge is recycled (to be mixed with the raw slurry/sludge) while the other portion enters a composting unit 900. In general, the composting unit comprises a dewatering unit for removing water from the matured sludge to form a dried filtrate; a mixing device for mixing structural material into the dried filtrate; a composting device for composting the dried filtrate. The composting unit fed into a dewatering screw press 730 to extract its free water, thereby forming a dried filtrate containing about 25% to 30% dry solid content. Free water extracted from the digestate (matured sludge) is reused in forming slurries with shredded raw sludge. Thereafter, the dried filtrate is delivered to a mixing device for mixing with structural material.

Mixing with structural material is carried out in order to facilitate composting of the dried filtrate, and in the present embodiment, mixing is carried out in a mixing screw 740 designed to evenly distribute the structural material into the filtrate to ensure proper aeration. The mixed filtrate is then laid out in heaps 750 on the floor of the composting building. The heaps may be arranged in any shape suitable for the building or land space allocated for the composting. To facilitate the aeration of the compost, the heaps are turned at regular intervals using windrow compost turners which moves and remixes the heaps, for example at intervals of 2 to 3 days.

To hasten the composting process, for example in land scarce areas or in areas where odour tolerance is low, aerated static pile composting may be carried out in which the heaps are composted in enclosed composting units having specially built floors which provide a constant supply of air to the compost. The floors of such composting units have aeration nozzles that are connected to air pipes. Air percolates through the dried filtrate while water sprinklers supply needed moisture to control the temperature of the composting process. Conditions in the composting unit, temperature and humidity for instance, are monitored and controlled by varying the amount of water supplied via the water sprinklers and the amount of air supplied by the aeration nozzles.

After approximately 4 weeks of composting, the heaps are converted from digested sludge to mature bio-compost that is suitable for use as fertilizers. The compost is screened in a compost segregator 760 to recover the structural material, which is then recycled with new dried filtrate from the screw press. Screened compost is stored in a bunker as bulk compost, and subsequently sent to a bagging plant where it is bagged in 25 kg bags and then palletised in 1 ton lots.

To summarize, the present invention provides device, a process and a system for digesting sludge anaerobically which offer the advantage of being carbon neutral, zero-effluent and economically sustainable. No wastewater is produced as all wastewater generated from the drying of the digestate (matured sludge) is being reused to form the slurry/sludge that is fed to the digesting tank. Odours are minimised as all areas of smell generation are subjected to extraction by fans via air ducts and processed for smell. This includes obnoxious gases generated from putrefying organic waste being processed prior to the digester and from the composting process which are extracted, scrubbed and treated in organic scrubbers. Noise generated from gas generators is rated to be not more than 55 decibels at the outer limits of the plant. Structural material used for composting is also entirely recycled, thereby not generating further waste material.

Although this invention has been described in terms of preferred embodiments, it has to be understood that variations and modifications may be made, without departing from the spirit and scope of this invention as set out in the following claims.

1. A device for digesting sludge anaerobically, comprising:
a digesting tank having an upper region and a lower region, and a reaction chamber for converting raw sludge into matured sludge arranged within the digesting tank,
an inlet for introducing sludge into the digesting tank,
at least one transfer pipe for channelling sludge from the lower region of the digesting tank to the upper region of the digesting tank, said at least one transfer pipe being arranged within the digesting tank and having at least a part of its length thereof arranged within the reaction chamber so that the at least one transfer pipe is in contact with sludge moving through the reaction chamber, thereby resulting in heat transfer from sludge moving in the at least one transfer pipe to sludge in the reaction chamber, and
an outlet arranged at the lower region of the digesting tank for discharging matured sludge from the digesting tank.

2. The device of claim 1, wherein the at least one transfer pipe is adapted to facilitate the transfer of heat from the sludge moving up the at least one transfer pipe to the sludge in the reaction chamber.

3. The device of claim 1, further comprising an actuating means for moving the sludge through the at least one transfer pipe.

4. The device of claim 3, wherein the actuating means comprises a screw pump.

5. The device of claim 1, wherein the at least one transfer pipe is supported within the reaction chamber.

6. The device of claim 1, further comprising a plurality of transfer pipes.

7. The device of claim 1, further comprising recycle means for re-introducing into the digesting tank a portion of matured sludge leaving the digesting tank through the outlet.

8. The device of claim 7, wherein the recycle means comprises recycle piping connected between the inlet and the outlet.

9. The device of claim 1, wherein the inlet is arranged at the lower region of the digesting tank.

10. The device of claim 9, wherein the inlet is connected to the at least one transfer pipe.

11. The device of claim 1, wherein the inlet is arranged at the upper region of the digesting tank.

12. The device of claim 1, wherein the digesting tank is adapted to maintain a vacuum in the reaction chamber.

13. The device of claim 1, wherein the reaction chamber is adapted to facilitate anaerobic digestion of the sludge.

14. The device of claim 1, further comprising mixing means for mixing raw sludge with matured sludge, thereby forming mixed sludge.

15. The device of claim 14, wherein the mixing means comprises a mixing region arranged in the lower region of the digesting tank, said mixing region being adapted to receive matured sludge from the reaction chamber and raw sludge from the inlet.

16. The device of claim 15, wherein the mixing means further comprises at least one screw pump having an inlet arranged at the lower region of the digesting tank.

17. The device of claim 16, further comprising a heat exchanger for heating the mixed sludge from the screw pump.

18. The device of claim 16, wherein the mixing device comprises a plurality of screw pumps.

19. The device of claim 1, further comprising a gas outlet arranged at the upper region of the digesting tank.

20. A process for treating sludge anaerobically, comprising:
introducing raw sludge into a device as defined in any one of Claim 1,
passing the sludge through the reaction chamber for a period of time sufficient for the slurry to be anaerobically digested, and
channelling a portion of the sludge from the lower region of the digesting tanks to the upper region of the digesting tank via the transfer pipe, and
discharging the digested sludge via the outlet.

21. The process of claim 20, wherein the reaction chamber of the device contains a micro-organism suitable for carrying out anaerobic digestion of the sludge.

22. The process of claim 20, further comprising screening the sludge to remove inorganic material prior to introducing the raw sludge into the device.

23. The process of claim 20, further comprising shredding the raw sludge prior to introducing the sludge into the device.

24. The process of claim 23, wherein the organic waste material is shredded to a size of less than 20 mm in diameter.

25. The process of claim 20, wherein the shredded organic waste material is mixed with water to form a raw slurry having a consistency of about 10% to about 20% of dry solid content.

26. The process of claim 20, wherein the raw slurry is mixed with digested sludge to form a mixed sludge, and introducing the mixed sludge into the digesting tank via the at least one transfer pipe in the digester.

27. The process of claim 26, wherein the mixed sludge is heated prior to being introduced into the digesting tank.

28. The process of claim 26, wherein the raw slurry is mixed with digested sludge in the ratio of at least 1 part raw slurry to 9 parts digested sludge.

29. The process of claim 20, wherein the reaction chamber is maintained at a temperature of about 50° C. to about 65° C.

30. The process of Claim 20, further comprising composting matured sludge that has been discharged from the digesting tank.

31. The process of claim 30, wherein composting comprises aerating and humidifying the mature sludge.

32. The process of claim 30, further comprising mixing the mature sludge with structured material.

33. The process of claim 30, further comprising dewatering the discharged mature sludge prior to composting.

34. The process of claim 33, wherein said dewatering is carried out until the dry solid content in the fermented sludge is about 25% to about 30%.

35. A system for digesting sludge anaerobically, said system comprising:
screening means for removing inorganic material from a raw waste,
shredding means for reducing the size of the raw waste,
mixing the raw waste into a slurry, and,
a device for digesting the raw slurry anaerobically as defined in claim 1.

36. The system of claim 35, wherein the screening means comprises a rotary screen having a screening size of approximately 150.

37. The system of claim 35, wherein the screening means comprises an electromagnet for removing ferrous material.

38. The system of claim 35, further comprising an electric generator unit for converting energy derived from the combustion of biogas gas produced from said anaerobic digestion into electricity.

39. The process of claim 38, further comprising a heat exchanger unit for transferring heat derived from said combustion to a portion of the raw sludge that is being channelled into the digesting tank.

40. The system of claim 35, further comprising a gas storage unit for storing biogas produced from the digestion of the sludge.

41. The system of claim 35, further comprising a composting unit for composting mature sludge that is discharged from the digesting tank.

42. The system of claim 41, wherein said composting unit comprises
a dewatering unit for removing water from the sludge,
a mixing screw for mixing wood chips with the sludge that has been treated in the dewatering unit, and a
and a composting device for converting the sludge that has been mixed with wood chips into compost.
